(19) United States
(12) Patent Application Publication
(10) Pub. No.: US 2006/0078476 A1
(43) Pub. Date: Apr. 13, 2006

(54) Electro-optical air purifier and emergency lights with ionizer

(76) Inventor: Se Kit Yuen, Hong Kong (CN)

Correspondence Address: ARTHUR KING MA, 1030 CORONADO DRIVE, ARCADIA, CA, 91007, US

(21) Appl. No.: 10/964,365

(22) Filed: Oct. 12, 2004

(51) Int. Cl. A61L 9/015

(52) U.S. Cl. 422/123; 96/224; 96/226

(57) ABSTRACT

The present invention provide an electro-optical air purifier with emergency night-light, which consists of one illuminant that is composed of one ozone light tube, two light sources and power. This product is characterized by its small size, light weight and integrated function of eliminating the virus and bacteria in the air while providing emergency lighting in the event of power shortage. It adopts both of the regular electrical power supply and any power provided by batteries. Said two power sources are replaceable and interchangeable.

### BACKGROUND OF THE PRESENT INVENTION

The present invention is relates to an air purifier and more particularly, to an air purifier with emergency night-light.

1. Field of Invention

In this modern society, illuminating apparatus is a must for families, hospitals, senior citizen centers, shopping malls, theaters, restaurants, office buildings, automobile manufacturers, elevators, various cars, ships, airplanes and trains. Meanwhile, air purifies are commonly used in nowadays. Especially problems of air pollution are getting worst as our society has become more industrialize, more depending on transportation vehicles, and more diseases are transmitted by air.

Most conventional air purifiers require normal indoor power supply such as 220V or 120 volt depending on geographical limitation, while few of those are designed mainly for transportation vehicles, which allow air purifier to function under a power supply generated by vehicles or any transportation vehicles batteries. Almost no conventional air purifier has an ability providing capacity for both indoor power supply and power supplied by batteries.

Moreover, most conventional air purifiers are designed to function as an independent unit without any ability of illumination. A consumer must purchase an additional illuminating equipment and install it next to an air purifier should he/she wants to have lighting available while enjoying a purified air. In additions, most conventional air purifiers are designed without any ability to provide any illumination during emergency period, which reduce consumers' abilities to escape should it be necessary during any unexpected situation.

Worsen still; most conventional air purifiers can only separate unwanted airborne particles from air to achieve so-called purification for air. However, this is a well-known fact that anions in the air are good for health. It would be beneficial should air purifiers can produce anions while separating unwanted airborne particles from air allows.

2. Description of Related Arts

### SUMMARY OF THE PRESENT INVENTION

A main object of the present invention is to provide an air purifier has an ability providing capacity for both indoor power supply and power supplied by batteries.

Another object of the present invention is to provide an air purifier has ability of illumination while purifying the air simultaneously or respectively.

Another object of the present invention is to provide an mean of illumination during emergency period, which increase consumers' abilities to escape should it be necessary during any unexpected situation.

Another object of the present invention is to provide an air purifier can produce anions while separating unwanted airborne particles from air flows.

Accordingly, in order to accomplish above objects, the present invention provides an air purifier with emergency night-light comprises:
a plurality of rectangular cylinder illuminant, and
at least one ozone light tube, and
a plurality of two lighting sources are assemble inside said illuminant, and
one of said two lighting sources is an emergency light bulb, which is illumining when power supplies are out while another one is a regular light bulb which is always on during normal usage and will be out at emergency and/or power supplies are out; and
a plurality of parabola-shaped reflector is attached at the inner portion of said illuminant jointly with a transparent front panel, which is connected to a terminal face of said reflector, said reflector is removable and is fixed at a front end portion of said illuminant;
a plurality of circuit board with electric initiative circuit, and two pieces of regular electrical connectors.

### BRIEF DESCRIPTION OF THE DRAWINGS

FIG. 1 is a side view of the present invention thereof;

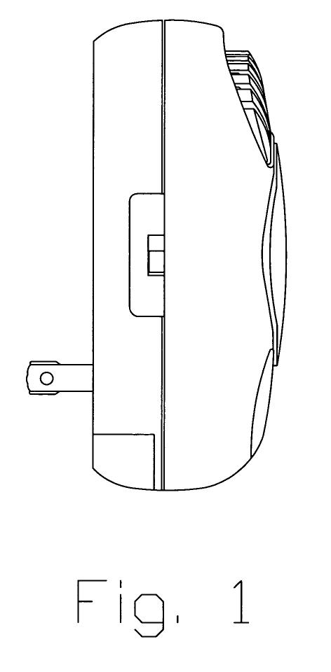

FIG. 2 is a vertical sectional view thereof;

FIG. 3 is a vertical view thereof;

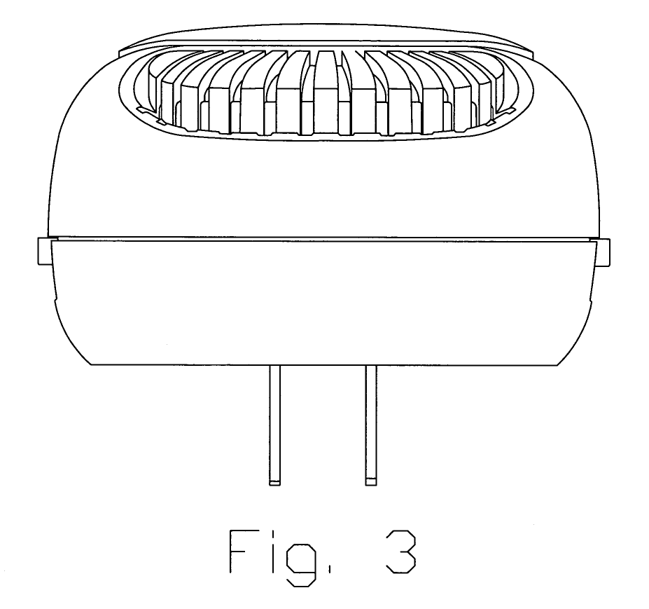

FIG. 4 is a top view thereof;

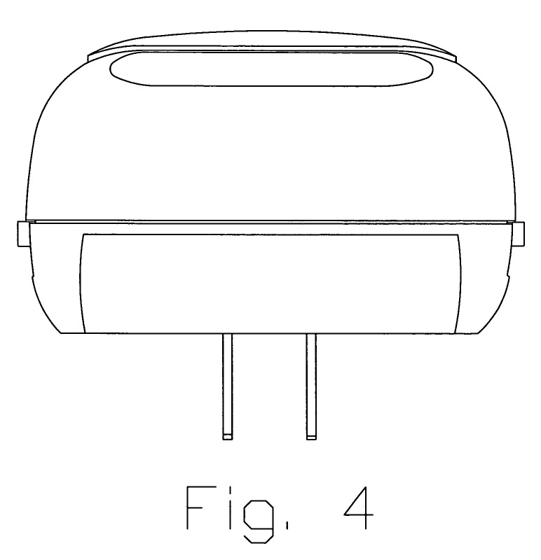

FIG. 5 is a frontal view thereof;

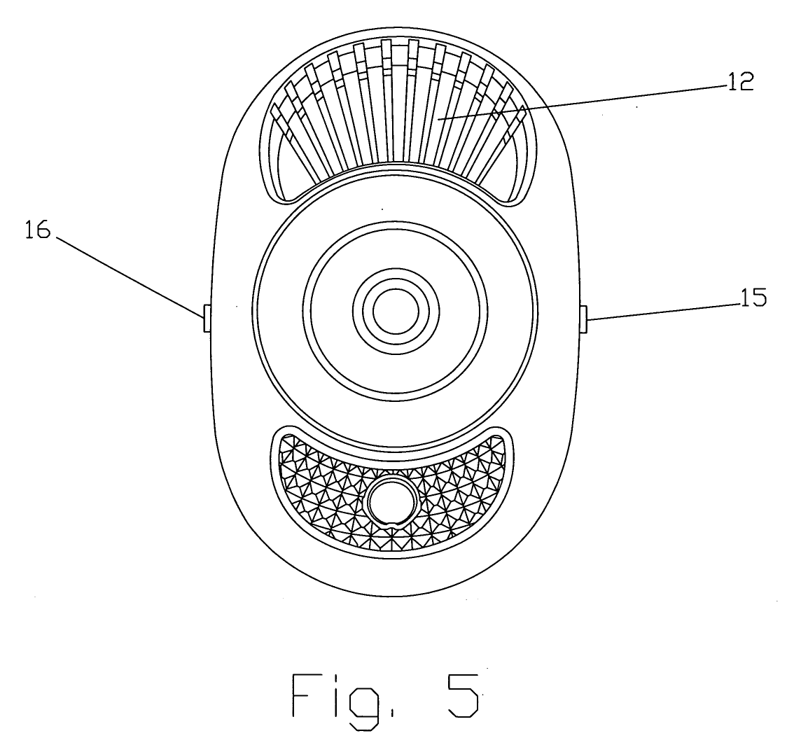

FIG. 6 is a rear view thereof;

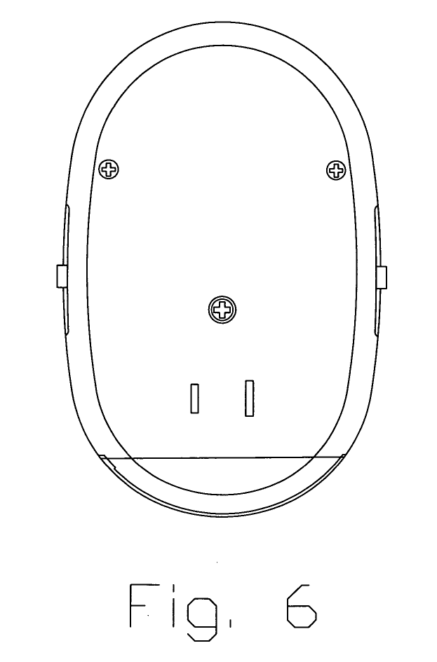

FIG. 7 is another side view thereof;

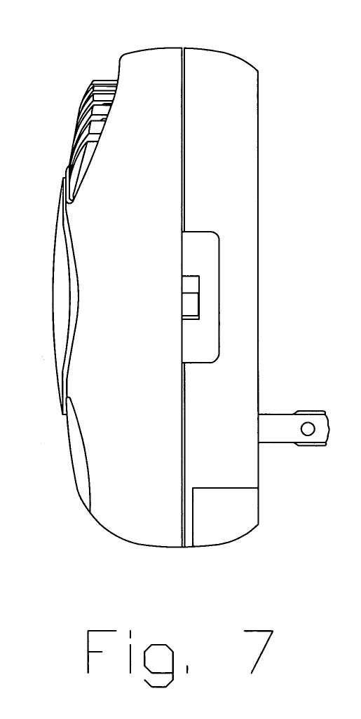

FIG. 8 is a perspective view thereof.

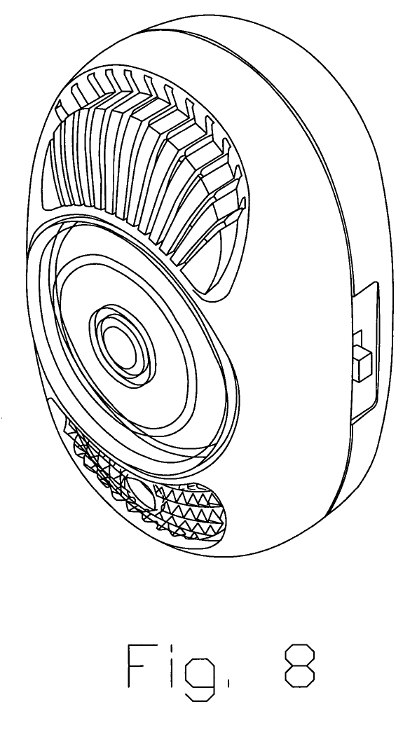

### DETAILED DESCRIPTION OF THE PREFERRED EMBODIMENT

This invention of electro-optical air purifier and emergency night-light has a rectangular cylinder illuminant (1). Inside said illuminant (1) there assembled two light sources, and one of them is an emergency light bulb (2) one of said two lighting sources is an emergency light bulb, which is illumining when power supplies are out while another one is a regular light bulb which is always on during normal usage and will be out at emergency and/or power supplies are out. Inside the illuminant there is also a parabola-shaped reflector (4) and one front panel (5) made of transparent material, which is connected to a terminal face of said reflector (4) from inside. Said reflector (4) is removable and is fixed to front end portion of said illuminant (1).

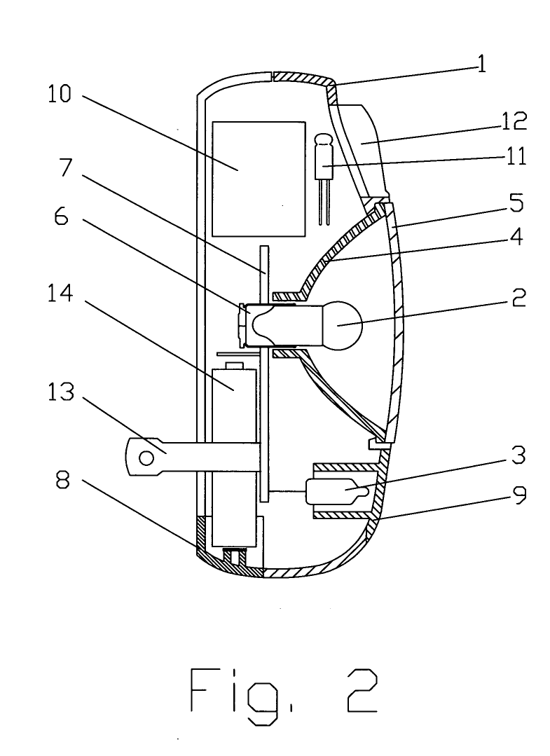

As shown in FIG. 2, a first source of light is an emergency light bulb (2) which is installed at a center portion of said reflector (4) in front of said illuminant (1). A bottom end of said emergency light bulb (2) is attached to a stand (6) in a flexible manner. Said stand (6) is fastened to a top portion of a circuit board (7), which is connected to another light source with a light bulb which is always on, at a bottom end portion (3); said light bulb which is always on (3) is affixed on a lampshade (9) which is made of semitransparent material. Said lampshade (9) is sealed and fixed to a bottom portion of a front end of said illuminant (1). Said circuit board (7) is connected to a photon initiator (10) on top. Said photon initiator directly activates said ozone light tube (11), and is capable of eliminating virus and bacteria in the air. Said ozone tube (11) is fixed to an upper inner surface of front end of said illuminant (1), which provides multiple breathers (12) to enhance elimination of virus and bacteria in the air by said ozone tube (11).

Said circuit board (7) is equipped with electric initiative circuit, and two pieces of municipal electrical connectors (13) made of metals on a lower end, which could be directly plugged into any regular electrical outlet to obtain power for said illuminant (1).

Said illuminant (1) has adoptability to two power sources (14), allowing power for the present invention convenience of its users so as to allow users to carry it around. When regular electrical power is out, said power source (14) will activate said electric initiative circuit device and said emergency light bulb (2) of said illuminant (1). Said power source (14) is installed on a top portion of a battery component (8) at a bottom portion of said illuminant (1). Said power source (14) has adoptability to any regular battery or rechargeable battery, which can be replaced.

Said illuminant (1) has a purifying switch (15) on its left side for user to turn on and off a power source for said ozone tube (11). There is a switch (16) at right side of said illuminant (1) to allow user to turn on or off said emergency light bulb (2).

What is claimed is:

1. An electro-optical air purifier and emergency night-light comprises:
a plurality of rectangular cylinder illuminant, and
at least one ozone light tube, and
a plurality of two lighting sources are assemble inside said illuminant, and
one of said two lighting sources is an emergency light bulb, which is illumining when power supplies are out while another one is a regular light bulb which is always on during normal usage and will be out at emergency and/or power supplies are out; and
a plurality of parabola-shaped reflector is attached at the inner portion of said illuminant jointly with a transparent front panel, which is connected to a terminal face of said reflector, said reflector is removable and is fixed at a front end portion of said illuminant; and
a plurality of circuit board with electric initiative circuit, and two pieces of regular electrical connectors.

2. An electro-optical air purifier and emergency night-light as recited in claim 1, wherein said parabola-shaped reflector and one front panel are made of transparent material, which is connected to a terminal face of said reflector from inside. Said reflector is removable and is fixed to front end portion of said illuminant.

3. An electro-optical air purifier and emergency night-light as recited in claim 1, wherein said illuminant has two power sources, which provide power for the present invention convenience of its users so as to allow users to carry it around. When regular electrical power is out, said power source will activate said electric initiative circuit device and said emergency light bulb of said illuminant. Said power source is installed on a top portion of a battery component at a bottom portion of said illuminant. Said power source has adoptability to any regular battery or rechargeable battery, which can be replaced. Said illuminant further comprises a purifying switch on its left side for user to turn on and off a power source for said ozone tube. Said illuminant further comprises a switch at right side of said illuminant to provide mean of turning on and off said emergency light bulb.

4. An electro-optical air purifier and emergency night-light as recited in claim 1, wherein aid circuit board is connected to a photon initiator on top. Said photon initiator directly activates said ozone light tube, and is capable of eliminating virus and bacteria in the air. Said ozone tube is affixed to an upper inner surface of front end of said illuminant, which provides multiple breathers to enhance elimination of virus and bacteria in the air by said ozone tube. Said circuit board further comprises:
a plurality of electric initiative circuit; and
at least two pieces of regular electrical connectors made of metals on a lower end portion of said illuminant providing mean of directly plugged into any regular electrical outlet so as to obtain power for said illuminant.

5. An electro-optical air purifier and emergency night-light as recited in claim 1, wherein said illuminant has adoptability to two power sources, allowing activation for said electric initiative circuit device and said emergency light bulb of said illuminant. Said power source is installed on a top portion of a battery component at a bottom portion of said illuminant. Said power source has adoptability to any regular battery or rechargeable battery, which can be replaced.

6. An electro-optical air purifier and emergency night-light as recited in claim 1, wherein said two power sources are replaceable and interchangeable.

7. A circuit board for electro-optical air purifier and emergency night-light, said circuit board is connected to a photon initiator on top;
said photon initiator directly activates said ozone light tube, and is capable of eliminating virus and bacteria in the air. Said ozone tube is affixed to an upper inner surface of front end of said illuminant, which provides multiple breathers to enhance elimination of virus and bacteria in the air by said ozone tube. Said circuit board further comprises:
a plurality of electric initiative circuit; and
at least two pieces of regular electrical connectors made of metals on a lower end portion of said illuminant providing mean of directly plugged into any regular electrical outlet so as to obtain power for said illuminant.

8. A circuit board for electro-optical air purifier and emergency night-light as recited in claim 7, wherein said illuminant has adoptability to two power sources, allowing activation for said electric initiative circuit device and said emergency light bulb of said illuminant. Said power source is installed on a top portion of a battery component at a bottom portion of said illuminant. Said power source has adoptability to any regular battery or rechargeable battery, which can be replaced. Said two power sources are replaceable and interchangeable.
